(19) United States
(12) Patent Application Publication
(10) Pub. No.: US 2008/0305474 A1
(43) Pub. Date: Dec. 11, 2008

(54) Method and Apparatus for Using SLC2A10 Genetic Polymorphisms for Determining Peripheral Vascular Disease in Patients with Type-2 Diabetes

(76) Inventors: Yuan-Tsong Chen, Taipei (TW); Lee-Ming Chuang, Taipei (TW)

Correspondence Address: LAW OFFICES OF LAI AND ASSOCIATES, P.C., 5800 RANCHESTER STE 200, HOUSTON, TX, 77036, US

(21) Appl. No.: 11/758,862

(22) Filed: Jun. 6, 2007

(51) Int. Cl. C12Q 1/68

(52) U.S. Cl. 435/6

(57) ABSTRACT

Recent data indicate that a loss-of-function mutation of the SLC2A10 gene causes arterial tortuosity syndrome (ATS) via upregulation of the TGF-β pathway in the arterial wall, a mechanism possibly causing vascular changes associated with diabetes. It is determined that SLC2A10 (Solute carrier family 2, facilitated glucose transporter, member 10) genetic polymorphism is associated with peripheral vascular disease (PVD) in patients with type 2 diabetes.

### FIELD OF THE INVENTION

The present invention relates generally to SLC2A10, more specifically, the present invention relates to method and apparatus for using SLC2A10 genetic polymorphisms for determining peripheral vascular disease in patients with type-2 diabetes.

### BACKGROUND

SLC2A10 gene is known. U.S. Pat. No. 6,849,728 to Bowden, et al. describes GLUT10: a glucose transporter in the type-2 diabetes linked region of chromosome 20Q12-13.1. In which GLUT 10 is described as an insulin-responsive glucose transporter gene located in the type-2 diabetes linked region of chromosome 20Q12-13.3. Isolated nucleic acids encoding the GLUT 10 glucose transporter, the encoded protein, antibodies that bind the protein, and methods of use are described herein.

Recent data indicate that a loss-of-function mutation of the SLC2A10 gene causes arterial tortuosity syndrome (ATS) via upregulation of the TGF-β pathway in the arterial wall, a mechanism that may cause vascular changes associated with diabetes.

Therefore, it is desirous to provide a method and apparatus for using SLC2A10 (Solute carrier family 2, facilitated glucose transporter, member 10) genetic polymorphism to determine peripheral vascular disease (PVD) in patients with type-2 diabetes.

### SUMMARY OF THE INVENTION

A method and apparatus for using SLC2A10 genetic variations in patients to determine a significant role in the development of PVD in type-2 diabetes is provided.

In the present invention, it is determined that the genetic polymorphisms of the SLC2A10 gene are associated with PVD in type-2 diabetic patients. it is further determined that the allele frequencies of the different SNPs and the resultant SNP haplotypes in diabetic patients with PVD be different from those without PVD. it is still further determined that the adjustment of the genetic risk calculations when taking into consideration the conventional vascular risk factors of PVD is necessary.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is an example of clinical characteristics of type 2 diabetic patients according to absence (−) or presence (+) of peripheral vascular disease (PVD). Italic indicates p<0.05, and bold highlights p<0.01 in accordance with some embodiments of the invention.

FIG. 2A is an example of a distribution of SNP genotypes and alleles of SLC2A10 in type 2 diabetes with and without PVD. with no adjustment for covariates in accordance with some embodiments of the invention.

FIG. 2B is an example of a distribution of SNP genotypes and alleles of SLC2A10 in type 2 diabetes with and without PVD with adjustment for covariates including age, duration of diabetes, sex, BMI, triglyceride, total cholesterol HbA1c, systolic blood pressure and smoking covariates in accordance with some embodiments of the invention.

FIG. 3 is an example of a sliding window analysis of haplotypes of two to fifteen neighboring SNPs for association with PVD in type 2 diabetic patients. The p-values are the minimum permuted p-value of a specific window size of haplotypes. Italic indicates P<0.05, and bold highlights P<0.01 in accordance with some embodiments of the invention.

FIG. 4A is an example of a multiple regression analyses of the SNP haplotypes of the SLC2A10 gene with PVD in type 2 diabetes having SNP haplotypes composed of 15 SNPs of the SLC2A10 gene were computed for their frequencies in accordance with some embodiments of the invention.

FIG. 4B is an example of a Multiple regression analyses of the SNP haplotypes of the SLC2A10 gene with PVD in type 2 diabetes analyzed for association with PVD in type 2 diabetes when adjusted for covariates including age, duration of diabetes, sex, BMI, triglyceride, total cholesterol HbA1c, systolic blood pressure and smoking in accordance with some embodiments of the invention.

FIG. 5 is an example of a key individual susceptible haplotypes associated with PVD in type 2 diabetic patients in accordance with some embodiments of the invention.

FIG. 6 is an example of showing significance of association of various SNPs and its haplotypes of the SLC2A10 gene with PVD in type 2 diabetic patients. The p-values are provided for each of the single SNP (vertical bar) and the haplotypes composed of 215 neighboring SNPs (horizontal line) in accordance with some embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to method and apparatus for using SLC2A10 genetic variations in patients to determine a significant role in the development of PVD in type-2 diabetes. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

Peripheral vascular disease (PVD), defined as lower extremity arterial atherosclerosis, is one of most common diseases of the arteries and is also a significant complication of type-2 diabetes. It has been recently recommended by the American Diabetes Association to be screened for patients with type-2 diabetes. See American Diabetes Association: PERIPHERAL ARTERIAL DISEASE IN PEOPLE WITH DIABETES, Diabetes Care 26:3333-3341, 2003, which is hereby incorporated herein by reference. PVD has been associated with conventional cardiovascular risk factors such as aging, smoking, hyperglycemia, hypertension and dyslipidemia. See both DEVELOPMENT OF MACROVASCULAR DISEASE IN NIDDM PATIENTS IN NORTHERN TAIWAN, Diabetes Care 16:137-143, 1993; and UKPDS 59: HYPERGLYCEMIA AND OTHER POTENTIALLY MODIFIABLE RISK FACTORS FOR PERIPHERAL VASCULAR DISEASE IN TYPE 2 DIABETES, Diabetes Care 25:894-899, 2002, both articles are hereby incorporated herein by reference.

However, the increased risk for atherosclerotic diseases in diabetic patients is only partially explained by classical risk factors. See REDUCING THE BURDEN OF DIABETES: MANAGING CARDIOVASCULAR DISEASE, Diabetes Metab Res Rev 15:186-196, 1999, which is hereby incorporated herein by reference. Although, a high heritability for an index of PVD, ankle-brachial blood pressure index (ABI), has been reported in twin studies in Caucasians suggesting an additional genetic factor might be involved in pathogenesis of PVD. See CONTRIBUTION OF GENETIC AND ENVIRONMENTAL INFLUENCES TO ANKLE-BRACHIAL BLOOD PRESSURE INDEX IN THE NHLBI TWIN STUDY, National Heart, Lung, and Blood Institute, AM J EPIDEMIOL 151:452-458, 2000, which is hereby incorporated herein by reference.

Search for genetic causes of PVD remains limited until recently, a genetic form of arterial tortuosity syndrome (ATS; OMIM 208050) has been reported to be caused by the loss-of-function mutations in SLC2A10 gene. See MUTATIONS IN THE FACILITATIVE GLUCOSE TRANSPORTER GLUT10 ALTER ANGIOGENESIS AND CAUSE ARTERIAL TORTUOSITY SYNDROME, Nat Genet 38:452-457, 2006, which is hereby incorporated herein by reference. SLC2A10 gene poses as a positional candidate susceptibility gene for type-2 diabetes mellitus (termed NIDDM3). See MOLECULAR CLONING OF A NOVEL MEMBER OF THE GLUT FAMILY OF TRANSPORTERS, SLC2a10 (GLUT10), LOCALIZED ON CHROMOSOME 20q13.1: A CANDIDATE GENE FOR NIDDM SUSCEPTIBILITY, Genomics 72:113-117, 2001, which is hereby incorporated herein by reference. Affected individuals are characterized by tortuosity of the large and medium-sized arteries, and often resulting in premature death at a young age. See RETROVIRAL OVEREXPRESSION OF DECORIN DIFFERENTIALLY AFFECTS THE RESPONSE OF ARTERIAL SMOOTH MUSCLE CELLS TO GROWTH FACTORS, Arterioscler Thromb Vasc Biol 21:777-784, 2001, which is hereby incorporated herein by reference. The features of vascular changes observed in ATS are similar to those described for Loeys-Dietz syndrome (LDS; OMIM 609192), characterized by increased pSmad2 and connective tissue growth factor (CTGF) expressions in the vascular walls, which are indicative of upregulation of transforming growth factor (TGF)-β signaling. The mechanisms by which mutations in SLC2A10 lead to TGF-β activation are unclear. Given the perinuclear localization of GLUT10 encoded by SLC2A10, it is suggested that a decrease in intracellular glucose transport might associate with a failure of glucose-mediated transcriptional upregulation of the decorin. Decrease in expression of decorin, a known proteoglycan inhibitor of TGF-β signaling, might lead to activation of TGF-β signaling.

Increased TGF-β activation plays a role in diabetic vascular complications. See THE CASE FOR TGF-β AS THE MAJOR MEDIATOR. J Am Soc Nephrol 15:S55-S57, 2004, which is hereby incorporated herein by reference. Further, TGF-β has been considered as a downstream effector of hyperglycemia-induced activation of protein kinase C. See THE PATHOBIOLOGY OF DIABETIC COMPLICATIONS. A UNIFYING MECHANISM, Diabetes 54:1615-1625, 2005, which is hereby incorporated herein by reference. In histopathology, the microangiopathic changes and fibrosis that are manifested in diabetic retinopathy, nephropathy and peripheral vascular disease have been shown to correlate with increased TGF-β signaling. See ROLE OF GROWTH FACTORS IN THE DEVELOPMENT OF DIABETIC COMPLICATIONS, Hormone Research 53:53-67, 2000, which is hereby incorporated herein by reference. Additionally, CTGF expression, which is seen to increase in the vessels of patients with ATS, is also found increased in kidney, myocardium, and aorta, in the same group of patients. This implies that CTGF might involve pathogenesis of both micro- and macrovascular diabetic complications. See both are hereby incorporated herein by reference REGULATION OF CONNECTIVE TISSUE GROWTH FACTOR ACTIVITY IN CULTURED RAT MESANGIAL CELLS AND ITS EXPRESSION IN EXPERIMENTAL DIABETIC GLOMERULOSCLEROSIS. J Am Soc Nephrol 11:25-38, 2000 and EXPRESSION OF CONNECTIVE TISSUE GROWTH FACTOR IS INCREASED IN INJURED MYOCARDIUM ASSOCIATED WITH PROTEIN KINASE C BETA2 ACTIVATION AND DIABETES. Diabetes 51:2709-2718, 2002, both publications are hereby incorporated herein by reference. Furthermore, impaired intracellular uptake or transport of monosaccharides, a function of GLUT10, might hinder glycosylation events important for the production of functional glycoproteins and proteoglycans that are essential structural components of the arterial wall and connective tissue in general. Taken the above results together, the SLC2A10 gene is a strong candidate gene for vascular complications in subject patients with type-2 diabetes.

The following is a practical example of experiments done relating to the present invention.

Subject Patients and Methods

Subject Patients and Phenotype Measurements

The subject patient under the experiment are of Han Chinese origin, without any known ancestors of other ethnic origin. A total number of 372 patients with type 2 diabetes diagnosed with the WHO criteria 1998 are recruited. For the WHO criteria 1998, see DEFINITION, DIAGNOSIS AND CLASSIFICATION OF DIABETES MELLITUS AND ITS COMPLICATIONS. PART 1: DIAGNOSIS AND CLASSIFICATION OF DIABETES MELLITUS PROVISIONAL REPORT OF A WHO CONSULTATION. DIABET MED 15:539-553, 1998, which is hereby incorporated herein by reference.

Body weight and height are measured to calculate body mass index (BMI). Seated blood pressure (BP) is measured after at least 5 min of resting. Demographic data and past medical history including cardiovascular, cerebrovascular and peripheral vascular diseases are documented. PVD herein is defined as a history of intermittent claudication or rest pain in association with absent foot pulses, gangrene, ischemic foot ulcers, lower extremity amputation due to ischemia, revascularization or an ABI <0.9 in any of the two limbs, using a handheld Doppler ultrasound (Medacord PVL, Medasonics, Fremont, Calif., USA) over brachial and dorsalis pedis or posterior tibial pulses.

The concentrations of plasma glucose, total cholesterol, and triglyceride are measured in fasting samples by an autoanalyzer (Hitachi 7250 special, Tokyo, Japan). HbA1c is measured with HPLC (CLC385, Primus Corporation, Kansas city, MO, USA).

SNP Genotyping

Genomic DNA is isolated using the PUREGENE™ DNA purification system (Gentra Systems, Minneapolis, Minn., USA). Selected SNPs of the SLC2A10 gene for genotyping are genotyped as previously reported. See AN ASSOCIATION STUDY OF GENETIC POLYMORPHISMS OF SLC2A10 GENE AND TYPE 2 DIABETES IN TAIWANESE POPULATION, Diabetologia 49: 1214-1221, 2006, which is hereby incorporated herein by reference. In total, 15 polymorphic markers including 14 SNPs (rs4810544, rs2425895, rs2143044, rs3092412, rs2235491, rs2425904, rs2425911, rs3091904, rs1059217, rs6066059, rs2179357, rs1003514, rs6018021 and rs6122518) and one (TGTGTGTGT)n microsatellite are genotyped as previously described. The failure of genotype has been certified by direct PCR sequencing.

Statistical Analysis

The Hardy-Weinberg equilibrium proportion (HWEP) test is carried out before conducting marker-trait association analyses. Re-sequencing experiments are undertaken to verify (or correct) the genotyping result of a SNP if it is not in Hardy-Weinberg equilibrium. The computing package SAS/Genetics is used for the single SNP association analysis, p-values are calculated from a Chi-square test. The sliding window approach is used to examine the associations of haplotypes in different window sizes of SNP combinations. P-values are computed by 1000 permutations, minimum permuted p-values for individual window sizes are reported. The association between PVD in type 2 diabetes and each common haplotype with or without adjusting for covariates is assessed through regression analyses. These association analyses are performed by the “Haplo.Stats” computing program. See SCORE TESTS FOR ASSOCIATION BETWEEN TRAITS AND HAPLOTYPES WHEN LINKAGE PHASE IS AMBIGUOUS. Am J Hum Genet 70:425-434, 2002, which is hereby incorporated herein by reference. P-values for testing significances of the regression parameters are also derived from 1000 permutations. Haplotypes with frequencies less than 5/(2*sample size)=0.0067 are grouped into the regression term “rare haplotype.

Results

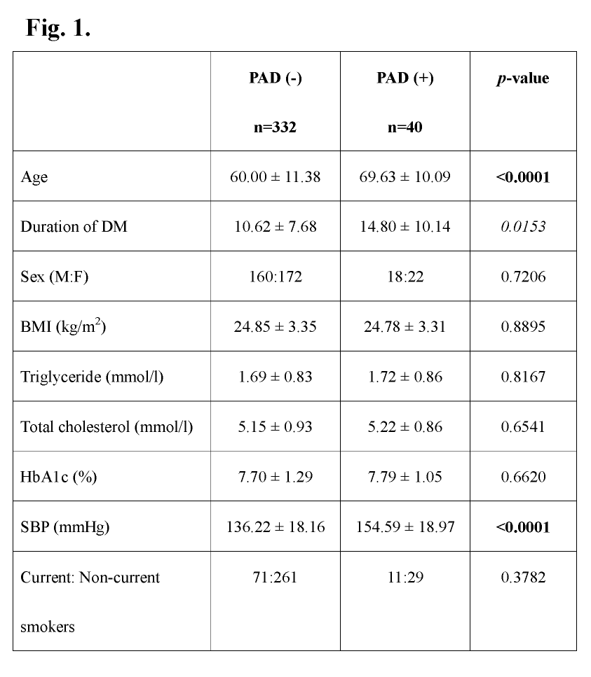

The characteristics of the study subject patients of type 2 diabetes with and without peripheral vascular disease (PVD) are summarized in FIG. 1 or Table 1. 40 (10.8%) of a total of 372 type 2 diabetic subject patients had a PVD. There are significant differences between PVD (−) and PVD (+) groups in age, duration of diabetes, and systolic blood pressure (SBP). No significant difference is found for other variables between the two groups.

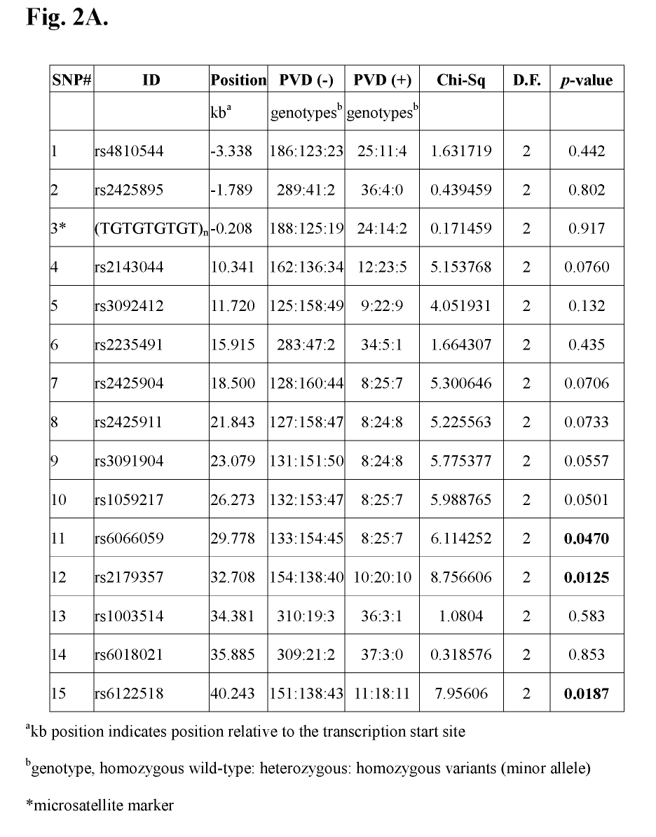

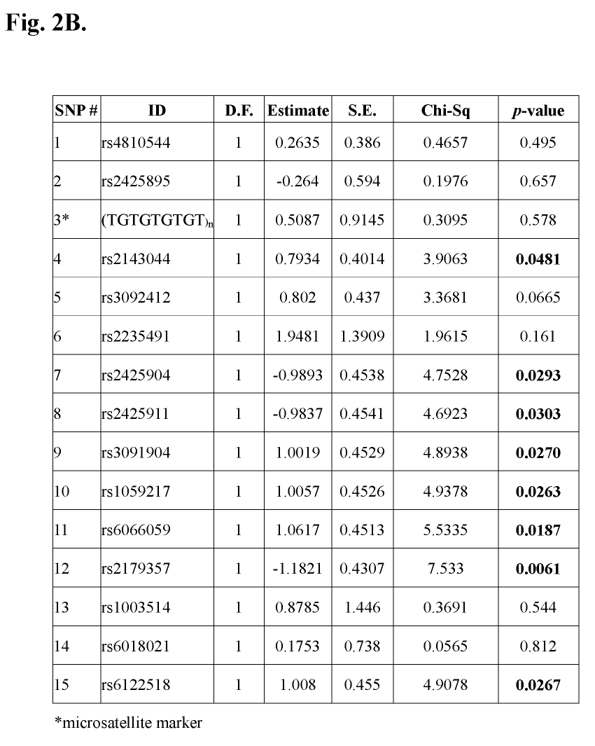

To study genetic association of the SLC2A10 gene with PVD in type 2 diabetic individuals, we first compared the difference in genotypic distribution between those with and without PVD (see FIG. 2A-2B or Table 2A-2B). Among the 15 polymorphic markers, there are significant differences in the genotype frequencies of 3 SNPs, i.e. rs6066059, rs2179357, and rs6122518, between the two groups (with a p-value <0.05, Fig. Table 2A). There is no correlation of the promoter (TGTGTGTGT)n microsatellite with PVD in type 2 diabetic individuals (FIG. 2A or Table 2A). With further adjustment for the covariates including age, duration of DM, sex, body mass index (BMI), triglyceride (TG), total cholesterol (TCH), HbA1c, systolic blood pressure (SBP) and smoking, there are 5 additional SNPs (rs2143044, rs2425904, rs2425911, rs3091904, and rs1059217) showing significant association with PVD (FIG. 2B or Table 2B).

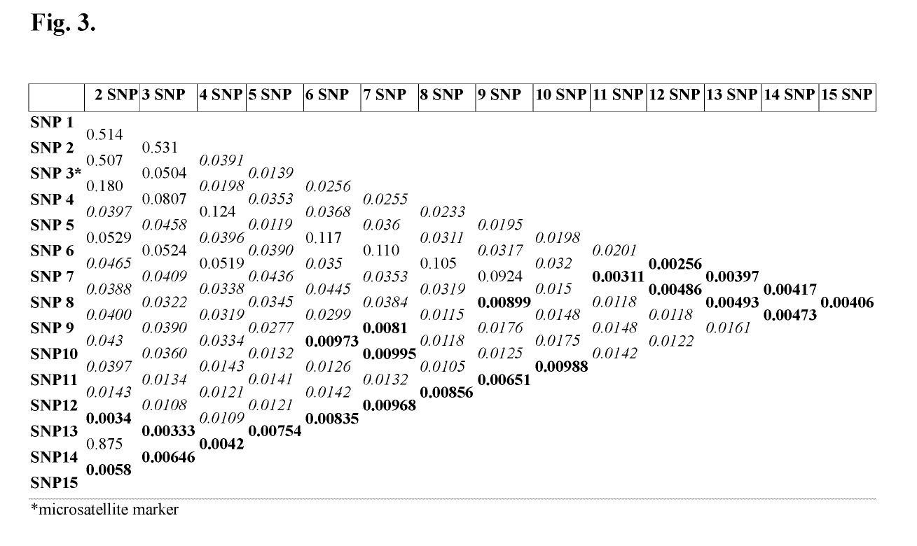

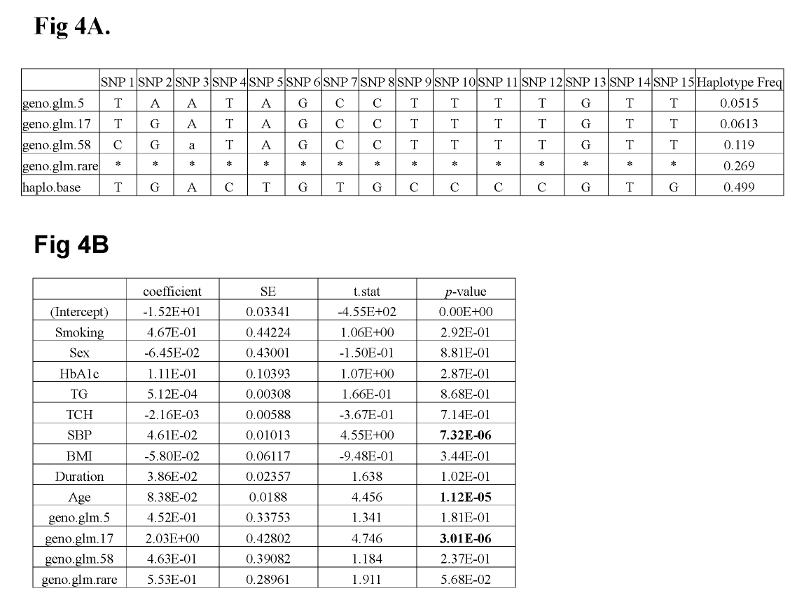

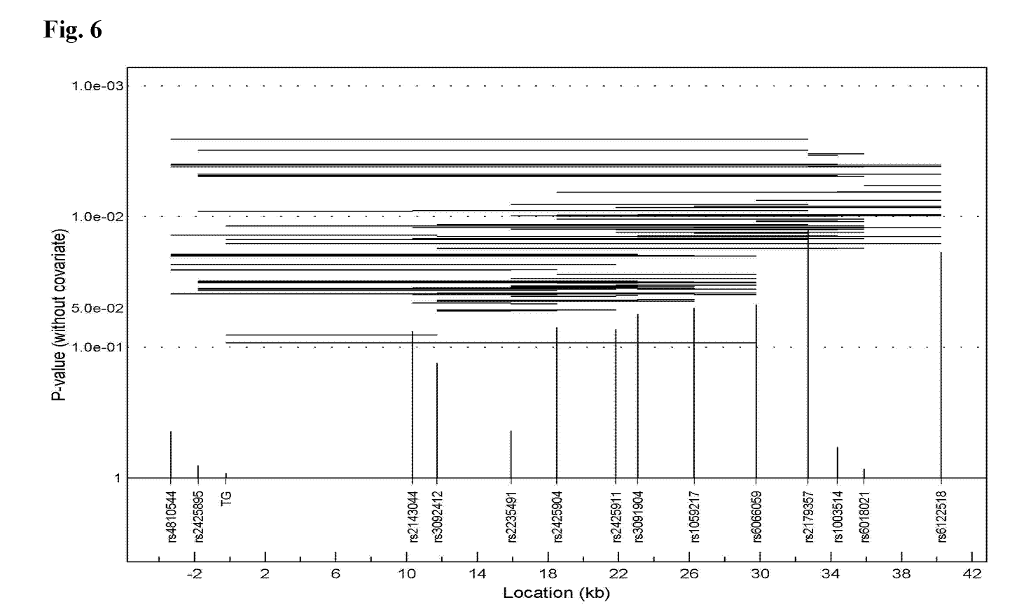

To further analyze the SNP haplotypes, sliding window analyses of haplotypes composed of 2˜15 neighboring SNPs are performed for association with PVD in type 2 diabetic patients (see FIG. 6). As described in the FIG. 3 or Table 3, the significance of association of respective SNP haplotype and PVD is analyzed with computing permutations. Using haplotypes constructed for adjacent SNPs, the association remains strong for the haplotype including all 15 SNPs across the gene (p=0.00406). Based on haplotype analyses, there are 4 major SNP haplotypes, composed of the 15 SNPs, with a cumulative haplotype frequency at 0.7308 (FIG. 4A or Table 4A). Multiple regression analysis is performed for PVD (FIG. 4B or Table 4B). In this model, in addition to the well-known risk factors, such as age (p=0.0000112) and systolic blood pressure (p=0.00000732), one specific SNP haplotype (geno.glm.17) is independently associated with PVD (p=0.00000301).

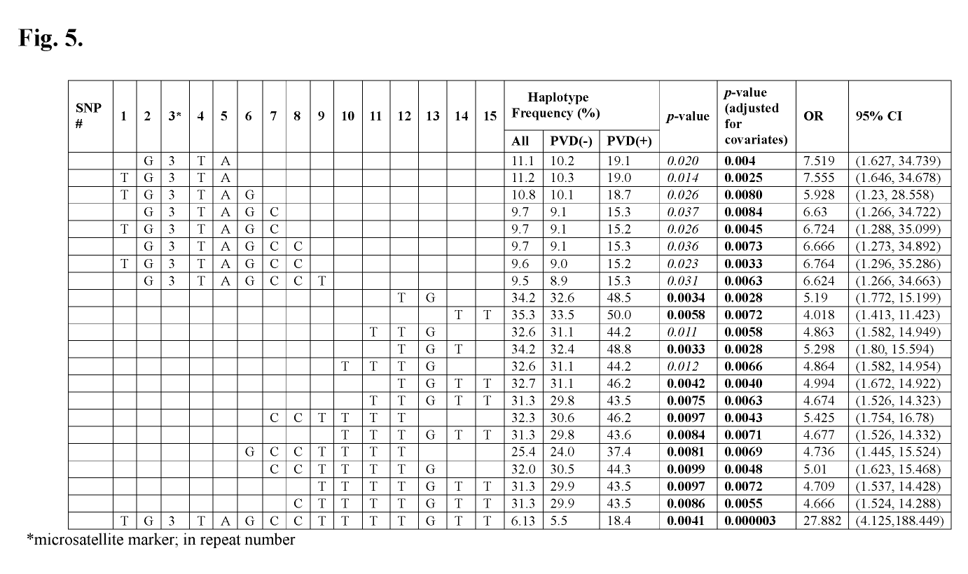

Referring to FIG. 5 or Table 5, to understand the effect of SNP haplotypes on PVD development, the nucleotide composition of the haplotypes are analyzed with a showing of significant association with PVD in type 2 diabetic individuals. Among all potential haplotypes, there are multiple susceptible SNP haplotypes with a significant odds ratio (OR ranges from 4.0 to 7.5, 95% CI ranges from 1.2 to 35.3, Fig. Table 5) to PVD in type 2 diabetes. These haplotypes are relatively common with a frequency ranging from 15.2 to 50.0% in patients with PVD vs. 8.9 to 33.5% in patients without PVD. In consistent with these various susceptible haplotypes, one specific SNP haplotype (geno.glm.17) composed of 15 SNPs confer susceptibility to PVD in type 2 diabetes mellitus is identified, with a highest OR=27.882 (95% CI 4.125, 188.449).

Direct DNA sequencing are performed for all exons and flanking intronic sequences of the GLUT10 gene in patients with PVD. No pathogenic mutations other than the aforementioned SNPs are found (data not shown).

Discussion

In this example, additional evidence of a role of SLC2A10 gene is provided in addition to the known risk factors including age and systolic blood pressure, on development of PVD in patients with type 2 diabetes. Among all possible haplotypes composed from the SNPs, 21 susceptible SNP haplotypes with an odds ratio up to 4.0˜7.5 [95% CI ranges from 1.2 to 35.3] with PVD are identified. It should be noted that the frequency of the identified haplotypes is relatively common, suggesting that the SLC2A10 gene might play a significant role in pathogenesis of PVD in type 2 diabetes mellitus.

Glucose transport by facilitated diffusion is mediated by a multigene family of several membrane glycoproteins termed glucose transporters whose expressions are tissue-specific. See FAMILY OF GLUCOSE-TRANSPORTER GENES IMPLICATIONS FOR GLUCOSE HOMEOSTASIS AND DIABETES. Diabetes 39:6-11, 1990, REGULATION OF GLUCOSE-TRANSPORTER GENE EXPRESSION IN VITRO AND IN VIVO, Diabetes Care 13:548-564, 1990, and THE GLUCOSE TRANSPORTER FAMILIES SGLT AND GLUT: MOLECULAR BASIS OF NORMAL AND ABERRANT FUNCTION JPEN J Parenter Enteral Nutr 28:364-371, 2004; the three publications are hereby incorporated herein by reference. Alterations of the level of expression of these glucose transporters by using transgenic or knock-out mouse models might result in changes in insulin sensitivity and modification of whole-body metabolism. See THE METABOLIC CONSEQUENCES OF ALTERED GLUCOSE TRANSPORTER EXPRESSION IN TRANSGENIC MICE. J Mol Med 74:639-652, 1996, which is hereby incorporated herein by reference. Subsequently, certain congenital defects of sugar metabolism have been demonstrated to be caused by aberrant transporter genes, such as haploinsufficiency of blood-brain barrier hexose carrier in the glucose transporter 1 deficiency syndrome, and Fanconi-Bickel syndrome by mutations in GLUT2. See GLUT-1 DEFICIENCY SYNDROME CAUSED BY HAPLOINSUFFICIENCY OF THE BLOOD-BRAIN BARRIER HEXOSE CARRIER Nat Genet 18:188-191, 1998 and MUTATIONS IN GLUT2, THE GENE FOR THE LIVER-TYPE GLUCOSE TRANSPORTER, IN PATIENTS WITH FANCONI-BICKEL SYNDROME, Nat Genet 17:324-326, 1997; both publications are hereby incorporated herein by reference. In addition, a malfunction of GLUT4 expression or regulation by HIV protease inhibitor appears to contribute to the insulin resistance syndrome. See INDINAVIR INHIBITS THE GLUCOSE TRANSPORTER ISOFORM GLUT4 AT PHYSIOLOGIC CONCENTRATIONS, AIDS16:859-863, 2002 and A STRUCTURAL BASIS FOR THE ACUTE EFFECTS OF HIV PROTEASE INHIBITORS ON GLUT4 INTRINSIC ACTIVITY, J Biol Chem 279:55147-55152, 2004, both publications are hereby incorporated herein by reference. Unlike the well-known glucose transporters, such as GLUT1, GLUT2 and GLUT4, the physiological function of the new member GLUT10 is relatively unclear. Recently, a genetic form of arterial tortuosity syndrome has been identified by a loss-of-function mutation in SLC2A10 gene with a pathology characterized by increased expression of pSmad2 and CTGF in the vascular walls. These findings suggest upregulation of TGF-β signaling occurs in the vascular walls, one of the final common pathways linking hyperglycemia and vascular complications in individuals with diabetes mellitus. In support of this notion, the present example provides a strong evidence of the genetic association of SLC2A10 PVD with patient having type 2 diabetes mellitus.

SLC2A10 gene encoding GLUT10 is a positional candidate susceptibility gene for type 2 diabetes mellitus, lies within NIDDM3 T2DM susceptibility region mapped on human chromosome 20q13.1 (10). However, up to this exemplified experiment, the evidence of association with type 2 diabetes is weak as studied in several populations including our own. There is no report analyzing the correlation of SLC2A10 gene and vascular complications in diabetes mellitus. It is noted that prevalence of PVD in type 2 diabetes is low in Asian populations when compared to Caucasians and Indians. The allele frequency of a commonly typed SNP, Ala206 Thr (rs2235491), is also different among populations, namely the Thr206 allele frequencies are 3˜5% in the Caucasians recruited from Europe and America are lower compared with those of 7.9˜9.4% in the diabetes and control populations recruited from Taiwan and 6˜7% in Finns. From the identified susceptible haplotype in this study (see FIG. 5 or Table 5), the Ala206 allele is susceptible while Thr206 allele is protective which may serve to explain the lower frequency of PVD in our population. Furthermore, previous study has provided evidence that Ala/Ala carriers exhibit higher fasting insulin level and the area-under-curve of the insulin levels after glucose loading. The Ala206 Thr might contribute to vascular complications in type 2 diabetes via the associated hyperinsulinemia in the Ala/Ala carriers. The present exemplified test urges the analyses in other independent populations to confirm the role of SLC2A10 in PVD development.

The association of the SNPs with PVD is strongest for the SNPs that are located on the 3′UTR and the downstream of SLC2A10 gene. Analyses of the sequences surrounding the SNP located on 3′UTR (rs1059217) revealed no conserved motif among different species to support the functional consequences. Other downstream SNPs, i.e. rs2179357, rs1003514, rs6018021, and rs6122518, are located far away from the SLC2A10 gene; for example, rs6122518 is located 10 k base pairs away from the 3′UTR. Whether the association is due to linkage disequilibrium with other genes downstream of SLC2A10 gene remained to be seen.

Ankle-brachial index (ABI) employed in this study is a non-invasive and objective tool which can be applied and easily adopted in clinical and epidemiological studies. With a cut-off at 0.9, the sensitivity and specificity of ABI for PAD are both over 90% (1.38). Subject patients with PVD or low ankle-brachial index (ABI), either symptomatic or subclinical, are found to have a higher mortality rate, partly due to comorbidity with coronary artery or cerebral vascular diseases. Despite several conventional risk factors known for macrovascular diseases, it is demonstrated that age, systolic blood pressure, and the genetic polymorphism of the SLC2A10 gene are independent risk factors for PVD. Whether SLC2A10 gene polymorphism contributes to other vascular complications and cardiovascular mortality remains for further investigation.

In conclusion, it is identified that relatively common SNP haplotypes of the SLC2A10 gene are associated with relative high OR to PVD in patients with type 2 diabetes. Data suggest that SLC2A10 genetic variations significantly contribute to PVD development in patients with type 2 diabetes.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A method for determining comprising the step of associating SLC2A10 gene with Peripheral Vascular Disease (PVD) in type-2 diabetic individuals.

2. The method of claim 1, wherein the SLC2A10 gene comprises a set of relatively common Single Nucleotide Polymorphism (SNP) haplotypes of the SLC2A10 gene.

3. The method of claim 1, wherein the SLC2A10 gene comprises anyone or all of the fourteen SNPs: rs4810544, rs2425895, rs2143044, rs3092412, rs2235491, rs2425904, rs2425911, rs3091904, rs1059217, rs6066059, rs2179357, rs1003514, rs6018021 or rs6122518.

4. The method of claim 1, wherein the SLC2A10 gene comprises gene comprises anyone or all of the three SNPs: rs6066059, rs2179357, or rs6122518.
